(19) United States
(12) Patent Application Publication
(10) Pub. No.: US 2025/0302386 A1
(43) Pub. Date: Oct. 2, 2025

(54) SYSTEMS AND METHODS FOR OPTIMIZING HEALTH CARE RESOURCES AND PATIENT MONITORING

(71) Applicant: LifeSignals, Inc., Milpitas, CA (US)

(72) Inventors: Saravanan Balasubramanian, Bangalore (IN); Shabeer Rasheed, Bangalore (IN); Midhun Mohan Sivam, Kerala (IN); Yvonne Gunning, Dalystown (IE)

(21) Appl. No.: 19/235,271

(22) Filed: Jun. 11, 2025

Related U.S. Application Data
(63) Continuation of application No. PCT/US23/84426, filed on Dec. 15, 2023.

(30) Foreign Application Priority Data
Dec. 15, 2022 (IN) 202241072638
Nov. 10, 2023 (IN) 202311077038

(51) Int. Cl. A61B 5/00

(52) U.S. Cl. CPC A61B 5/6833; A61B 5/0002

(57) ABSTRACT

The present disclosure provides a healthcare platform that delivers monitoring services to patients in clinical settings and non-clinical settings. An example method for monitoring physiological data is described. The method comprises (a) contacting a surface of a subject with a patch, where the patch is configured to (1) monitor, with aid of one or more sensors operably coupled to the patch, physiological data from the subject, and (2) receive, at an electronic module in communication with the one or more sensors, the monitored physiological data; and (b) wirelessly transmitting the received physiological data to two or more different types of devices.

### CROSS-REFERENCE

The present application claims the priority of Indian Patent Application No. 202241072638, filed Dec. 15, 2022, Indian Patent Application No. 202311077038, filed Nov. 10, 2023, and PCT application number PCT/US2023/084426, filed on Dec. 15, 2023, published as WO2024/130200 on Jun. 20, 2024, each of which is incorporated herein by reference in its entirety.

### BACKGROUND

Monitoring physiological conditions of patients has been an important component of health care. Although the monitoring can be performed periodically by healthcare providers, the task is being handled by electronics that connect the patient to a computerized system for autonomous data processing, storage, presentation and retrieval. The autonomous monitoring of physiological condition has also become an important part of everyday life with the advent of the quantified-self movement.

Remote health monitoring including near-real time monitoring makes it easier and cost effective to monitor the health of vast populations. Wireless systems are desirable to enable remote health monitoring. Conventional wireless health monitoring systems can be bulky, expensive, have inadequate wireless link reliability, or have high power dissipation which limits their applications, for example, to monitoring a wide range of physiological parameters in high volumes for large populations. In some instances, health monitoring systems can inadequately account for concurrent use with other forms of medical equipment such as defibrillators.

### SUMMARY

Globally, over 65 percent of hospital patients and over 90 percent of post-acute care patients are monitored manually and not continually. Many vital sign changes are missed during spot checks which often occur in four-to-six-hour intervals. A UK national audit of adult in-hospital cardiac arrests showed that 57 percent occurred on the patient wards and only five percent in the Intensive Care Unit (ICU) where patients are monitored continuously. Most patients who end up in cardiac arrest or critical care, don't suddenly deteriorate but rather present with earlier vital signs that show abnormal trends.

Often, wireless health monitoring systems may be directed to achieving a fixed application or objective (e.g., monitoring heart rate, monitoring temperature, etc.), and the associated hardware (e.g., sensors, etc.) and software (e.g., measurement parameters and signal processing, etc.) may be engineered specifically to achieve only the fixed application. For example, such systems may be restricted to only a single application or objective. These systems may not be compatible with other, different applications or objectives, and lack significant flexibility in use. In most cases, the entire system (e.g., device, program, etc.) may have to be deconstructed and/or reconstructed to accommodate the different applications, including making hardware modifications and software modifications. Such retroactive procedures can be very costly, inefficiently, and time-consuming.

The present disclosure provides a healthcare platform that may deliver healthcare to mass populations. The systems and methods provided herein may deliver hospital-grade active and continuous monitoring of patients. In some embodiments, the platform may provide hospital-grade active monitoring services after the patient is released from hospital. One or more wearable patches may facilitate monitoring operations, where the patient is untethered. The platform may employ one or more wireless access points within a facility to facilitate real-time or near real-time data transmission. In some embodiments, the platform may be alert-based to reduce the amount of attention required from healthcare providers. The platform may provide one or more graphical user interfaces (GUIs) that integrate and present user information obtained from multiple sources. The systems and methods provided herein may enable efficient active monitoring and treatment for a mass population. By optimizing health care resource allocation, the systems and methods provided herein may reduce the amount of time that healthcare providers spend on monitoring patients and thus, increase the efficiency and efficacy in healthcare.

An aspect of the present disclosure is a method for monitoring physiological data. The method comprises (a) contacting a surface of a subject with a patch, wherein the patch is configured to (1) monitor, with aid of one or more sensors operably coupled to the patch, physiological data from the subject, and (2) receive, at an electronic module in communication with the one or more sensors, the monitored physiological data; and (b) wirelessly transmitting the received physiological data to two or more different types of devices.

In some embodiments, the two or more different types of devices comprise at least two of a mobile device, a data collection device, and a patient monitor.

In some embodiments, the patch is configured to communicate with the data collection device via Wi-Fi, MBAND and, and/or UWB. In other embodiments, the patch is configured to communicate with the mobile device via Wi-Fi. In other embodiments, the patch is configured to communicate with the patient monitor via Wi-Fi, MBAND, and/or UWB.

In some embodiments, the data collection device is further configured to communicate with the mobile device and/or the patient monitor. In other embodiments, the data collection device is further configured to communicate with an external server.

In some embodiments, the patch is configured to communicate with the two or more devices using different communication schemes. In other embodiments, the patch is configured to communicate with the two or more devices using a same communication scheme.

In some embodiments, the two or more different types of devices receive physiological data transmitted from a plurality of patches.

In some embodiments, the one or more sensors are configured to measure one or more of electrocardiogram (ECG), blood saturation (SpO2), heart rate (HR), respiratory rate (RR), heart rate variability (HRV), blood pressure, body temperature, posture, attitude, acceleration, or motion.

Another aspect of the present disclosure is a system for monitoring physiological data from a plurality of patients. The system comprises (a) a plurality of patches configured to monitor the physiological data from a plurality of patients; and (b) a data collection device in wireless communication with the patch, the data collection device configured to receive the monitored physiological data from the plurality of patches, and transmit to an external server.

Beneficially, the patient monitoring platform provided herein may have sufficient flexibility to accommodate and enable the monitoring of different activities, even those uncontemplated at the time of manufacture of the hardware portion of the wireless platform. Any user, such as individuals, entities, manufacturers, healthcare professionals, medical operators, and others, may design and engineer an application utilizing any combination of a plurality of sensors included in the wireless platform to achieve an objective, including clinical objectives. Such applications may be created, prior to, simultaneously, or subsequent to manufacture of the hardware portion of the wireless platform. The patient monitoring platform may comprise a processor and/or electronic module capable of controlling the plurality of sensors in the wireless platform to comply with the requirements and instructions of the different applications executed by an external device in communication with the wireless platform via a communication interface of the wireless platform.

It shall be understood that different aspects of the present disclosure can be appreciated individually, collectively, or in combination with each other. Various aspects of the disclosure described herein may be applied to any of the particular applications set forth below. Other objects and features of the present disclosure will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference in their entirety.

### BRIEF DESCRIPTION OF THE DRAWINGS

The technical features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of the following.

FIG. 1 illustrates comparison between a tethered patient monitoring system and an example of wireless patient monitoring platform disclosed herein.

FIG. 2 illustrates an example of wireless patient monitoring platform implemented in hospitals.

FIG. 3 illustrates comparison between existing biosensors and an example of improved biosensors.

FIG. 4 illustrates a communication schematic between patients and hospitals, clinics, and nursing homes using an example of patient monitoring platform disclosed herein.

FIG. 5 illustrates an architecture of an example of patient monitoring platform.

FIG. 6 illustrates an example of patient monitoring platform comprising a wearable patch with multiple radios for wireless communication.

FIG. 7 illustrates an example of multi-patient monitoring platform for wireless communication.

FIG. 8 illustrates another example of multi-patient monitoring platform for wireless communication.

FIG. 9 illustrates another example of multi-patient monitoring platform for wireless communication.

FIG. 10 illustrates user hierarchy of an example of patient monitoring platform.

FIG. 11A illustrates an example of graphical user interface (GUI) of a patient monitoring platform for multi patient view.

FIG. 11B illustrates an example of GUI of a patient monitoring platform for expanded patient view.

FIG. 11C illustrates an example of GUI of a patient monitoring platform for alerts view.

FIG. 12 illustrates an example of GUI of service provider admin dashboard for user management.

FIG. 13 illustrates an example of GUI of service provider admin dashboard for clinical facility management.

FIG. 14 illustrates an example of GUI of a settings landing page in a clinical facility admin dashboard.

FIG. 15 illustrates an example of GUI of clinical facility admin dashboard for user management.

FIG. 16 illustrates an example of GUI of clinical facility admin dashboard for patient group customization.

FIG. 17 illustrates an example of GUI of clinical facility admin dashboard for digital twin of locations.

FIG. 18 illustrates an example of GUI of clinical facility admin dashboard for clinical alert settings.

FIG. 19 illustrates an example of GUI of clinical facility admin dashboard for technical alert settings.

FIG. 20 illustrates an example of GUI of a settings landing page in a clinical facility nurse dashboard.

FIG. 21 illustrates an example of GUI of a clinical facility nurse dashboard for admitting patients.

FIG. 22 illustrates an example of GUI of a clinical facility nurse/physician dashboard for current patient view.

FIG. 23 illustrates an example of GUI of a clinical facility nurse/physician dashboard for alert patient view.

FIG. 24 illustrates an example of GUI of a nurse/physician monitoring dashboard for tile view with waveforms of physiological parameters.

FIG. 25 illustrates an example of GUI of a nurse/physician monitoring dashboard for tile view of custom patients.

FIG. 26 illustrates an example of GUI of a nurse/physician monitoring dashboard for tile view without waveforms of physiological parameters.

FIG. 27 illustrates an example of GUI of a nurse/physician monitoring dashboard for tile view with blood pressure.

FIG. 28 illustrates an example of GUI of a nurse/physician monitoring dashboard for group alert view.

FIG. 29 illustrates an example of GUI of a nurse/physician monitoring dashboard for event listing.

FIG. 30 illustrates an example of GUI of a nurse/physician monitoring dashboard for alert destination configuration.

FIG. 31 illustrates an example of GUI of a nurse/physician monitoring dashboard for trend display.

FIG. 32 illustrates an example of GUI of a settings landing page in an alert-based patient monitoring dashboard.

FIG. 33 illustrates an example of GUI of an alert-based patient monitoring dashboard with patient alerts.

FIG. 34 illustrates an example of a wearable patch with a dual radio for wireless communication.

FIGS. 35A, 35B, and 35C illustrate examples of GUI that provide feedback and guide the patient to wear a patch.

FIGS. 36A and 36B illustrate examples of GUI that guides the patient to wear a patch.

### DETAILED DESCRIPTION

While various embodiments are shown and described herein, it will be obvious to those skilled in the art that such embodiments are provided by way of example only. Numerous variations, changes, and substitutions may occur to those skilled in the art without departing from devices, systems and methods disclosed herein. It should be understood that various alternatives to the embodiments described herein may be employed.

Unless otherwise defined, all technical terms used herein have the same meaning as commonly understood by one of ordinary skill in the art to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated.

### Overview

The technologies disclosed herein may support various health monitoring procedures. Examples of the health monitoring procedures include, but are not limited to: multi-day live monitoring where a user may be monitored by a remote healthcare provider or a family member; in-patient monitoring where data is continuously displayed on patient monitors to facilitate patient monitoring; ambulatory outpatient monitoring where an ambulatory staff member can monitor physiological signals and correlate the physiological signals with symptoms; Holier monitoring where a person's various electrical activity of cardiovascular system can last more than 24 hours; event monitoring where an event can be flagged either by a patient or others, e.g. by pressing a button and few minutes of data may be transmitted on occurrence of an event.

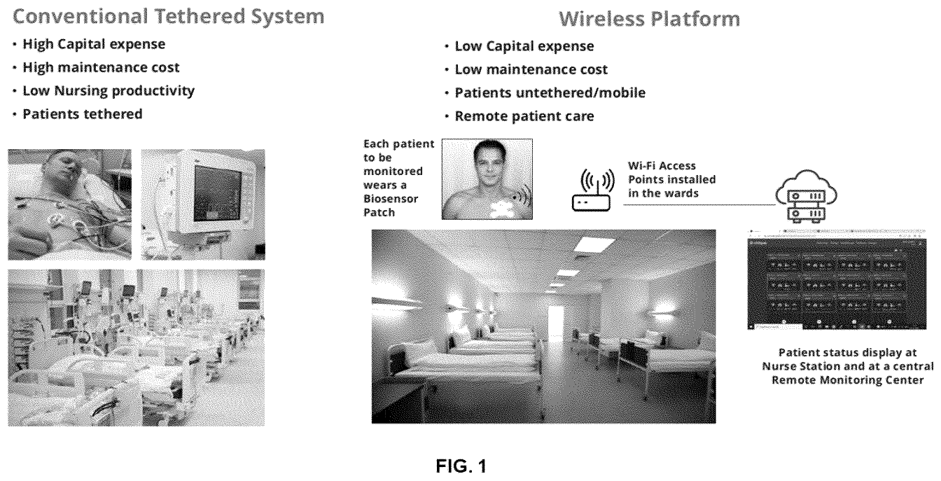

FIG. 1 illustrates comparison between a tethered patient monitoring system and an example of wireless patient monitoring platform. A majority of hospitals and clinics nationwide and even worldwide use tethered patient monitoring systems, wherein patients are connected with wired physiological sensors and patient monitors. In a hospital with multiple wards and hundreds of patients, tethered patient monitoring systems often suffer from high capital expense, high maintenance cost, and low nursing productivity. The technologies disclosed herein resolves these drawbacks. The wireless patient monitoring system disclosed herein provides a wireless wearable patch that includes multiple biosensors. Each patient may be monitored wearing a patch, irrespective the patient's physical location. The biosensors within the wearable patch may monitor and collect physiological parameters of the patient, and wirelessly transmit the signals to a patient care center. For example, patients admitted to hospitals may wear a patch for continuous monitoring. Without being confined to bedside, patients can have substantial flexibility in mobility. This also allows visitors to interact with the patient without technology getting in the way. Moreover, the wireless patient monitoring system provides patients and family members peace of mind knowing that monitoring is constant—even when the patient is out of their room. Patient mobility may help improve patient outcomes and reduce length of stay, which may lower costs and elevate patient satisfaction. The physiological signals collected by the wearable patch may be transmitted via Wi-Fi access points installed in the patient wards and displayed at a nurse station and/or a central remote monitoring center. Therefore, the wireless patient monitoring system may achieve flexible patient care while having low maintenance cost and low capital expense.

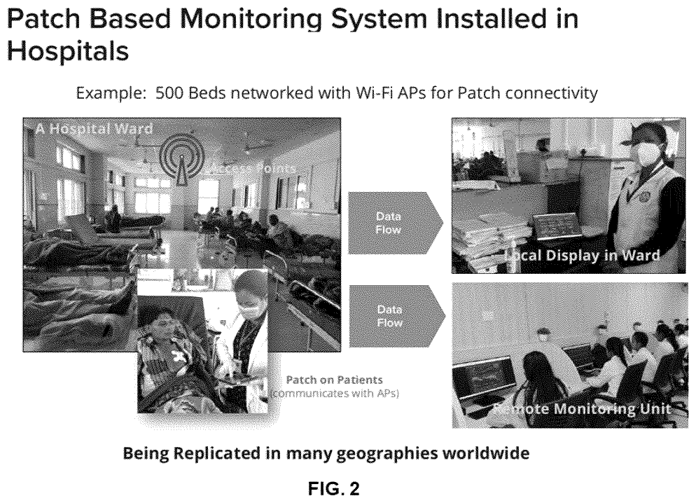

FIG. 2 illustrates an example of wireless patient monitoring platform implemented in hospitals. As illustrated, patients within a hospital may wear patches that continuously monitor their physiological parameters. Wards in the hospital may be installed with Wi-Fi access points. The wearable patches may collect physiological signals from the patients and transmit them to nurse stations in charge of the wards and a remote monitoring unit. Hence, the wireless patient monitoring platform can provide multi-patient monitoring on a large scale. The implementation of the wireless patient monitoring platform as illustrated in FIG. 2 is replicated in a variety of geographics worldwide.

The platform may allow real-time or near real-time monitoring of patients at multiple locations, including hospitals, skilled nursing facilities, clinics, ambulance, public transportations with network connection (e.g., train, bus, airplane), and patient homes. The platform may also provide a multi-patient monitoring dashboard for healthcare providers to review multiple patients simultaneously, regardless of where the patients are located. When patients are on the move, for example, from accident site, getting admitted to a hospital, to post discharge from the hospital, the platform as described herein may allow continuous patient monitoring.

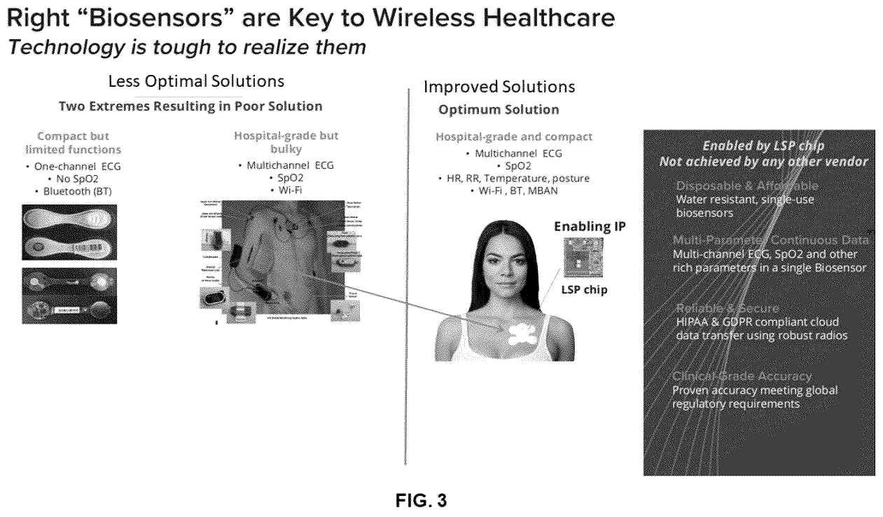

FIG. 3 illustrates comparison between existing biosensors and an example of improved biosensors. A majority of existing wireless biosensors have limitations. Some of the wearable patches are compact but with limited functions, for example, contain only a one-channel electrocardiogram (ECG) sensor and without oxygen saturation (SpO2) sensor. Some patches have better performance, i.e., contain multiple sensors (e.g., multi-channel ECG sensors and SpO2 sensor), but are bulky. The wireless patient monitoring platform disclosed herein comprise improved biosensors compacted into a wearable patch. In some embodiments, the wearable patch may comprise one or more sensors that monitor multi-channel ECG, SpO2, heart rate (HR), respiratory rate (RR), heart rate variability (HRV), blood pressure, body temperature, posture, attitude, acceleration, and/or motion. The one or more sensors in the wearable patch may provide clinical-grade accuracy that meets global regulatory requirements. The size of the wearable patch may be small enough that patients can wear them without being limited in mobility.

The wearable patch may comprise components that realize a variety of wireless communications, including Wi-Fi, Bluetooth®, radio, and/or medical body area network. In some instances, a selected wireless communication technique may be employed based on one or more circumstances of use. In some instances, the circumstances of use may be sensed with aid of one or more bio-sensors, or availability or data pertaining to the availability of status of the wireless communications. Optionally, the communication channel may be selected based on a sensed condition, such as a sensed environmental condition, sensed power level condition (e.g., battery level), sensed data level condition (e.g., needed bandwidth), locations of patches relative to relay, sensed physiological condition, requirements or instructions from an application of a relay, or any other conditions. In some instances, the wireless communication technique may be automatically selected with aid of a processor without requiring human intervention. Alternatively, an individual may select the wireless communication technique. The wireless communication may allow reliable and secure data transfer compliant with Health Insurance Portability and Accountability Act (HIPAA) and General Data Protection Regulation (GDPR).

The wearable patch may comprise a plurality of attributes that allow it to be worn by patients on large scales and in flexible patient care settings. For example, the wearable patch may be disposable after single use or with a pre-determined use life. The pre-determined use life may be one day, two days, three days, four days, five days, fix days, seven days, eight days, nine days, ten days, twelve days, fifteen days, twenty days, twenty five days, or thirty days. In some instances, an entirety of the patch may be disposable, or a portion of the patch may be disposable while a portion is reusable. A reusable portion of a patch may be attachable and/or separatable from a disposable portion.

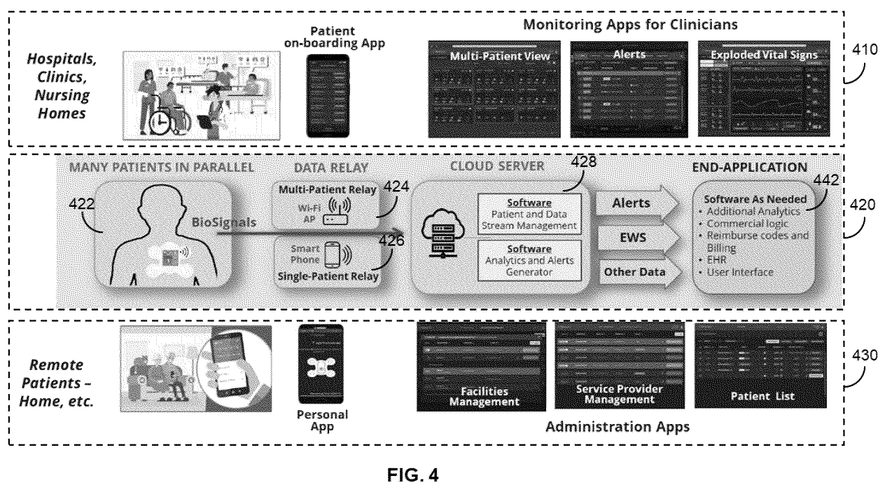

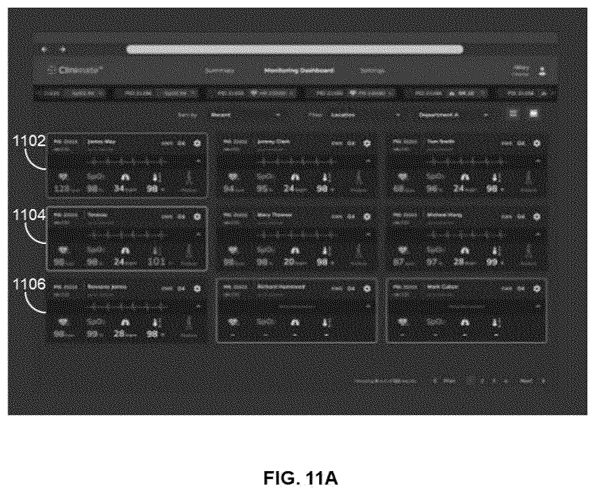

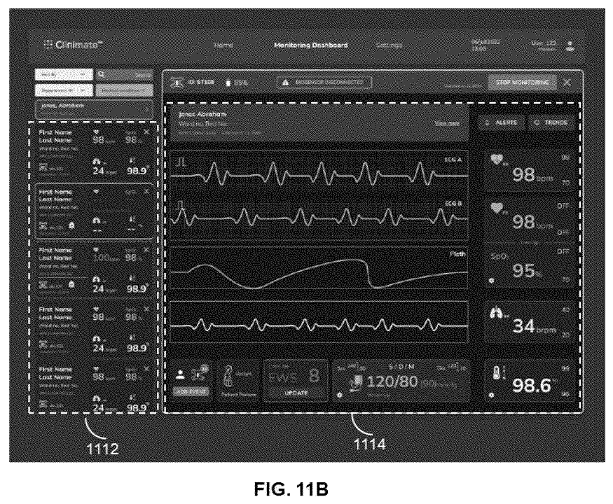

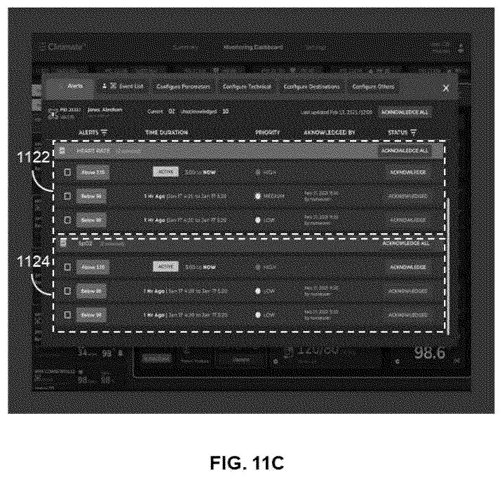

FIG. 4 illustrates a communication schematic between patients and hospitals, clinics, and nursing homes using an example of patient monitoring platform. The wireless patient monitoring platform as described herein allows real-time in-patient and output-patient monitoring, regardless patients' physical locations. As illustrated, the wireless patient monitoring platform allows simultaneous multi-patient monitoring in both clinical and home settings (see 420). Each patient may wear a patch containing a plurality of biosensors that collect physiological signals (see 422) that are wirelessly transmitted. When a patient is admitted to hospitals, clinics, or nursing homes, medical staff may assist the patient's admission via a patient on-boarding application. As the hospital may admit multiple patients, each of them may wear a patch that monitors their health conditions. The physiological signals collected from the wearable patch may be wirelessly transmitted via a single- or multi-patient data collection device, for example, relay (e.g., Wi-Fi access points, see 424) to a server (see 428). The server may comprise software that processes the physiological signals, generate alerts if the patient's health condition deteriorates, and manages patient data stream. The generated alerts, early warning signals/scores (EWS) that indicate early signs of clinical deterioration of the patient, and other data that is valuable to patient management may be displayed and/or further processed in end applications. For example, when patients are admitted in hospitals, clinics, or nursing homes, health care providers (e.g., clinicians, nurses, specialists) may be allowed to monitor the health conditions of multiple patients (see 410) via one or more monitoring applications. In some embodiments, the monitoring applications may provide GUIs that allow health care providers to review real-time physiological parameters of multiple patients simultaneously (e.g., multi-patient view), review patients that have active alerts with respect to one or more parameters (e.g., alerts view), and review the detailed information (e.g., waveforms) of one or more parameters of a particular patient (e.g., exploded vital sign view). More examples of GUI and detailed description thereof will be provided below, in accordance with FIGS. 11A-11C, and 22-31.

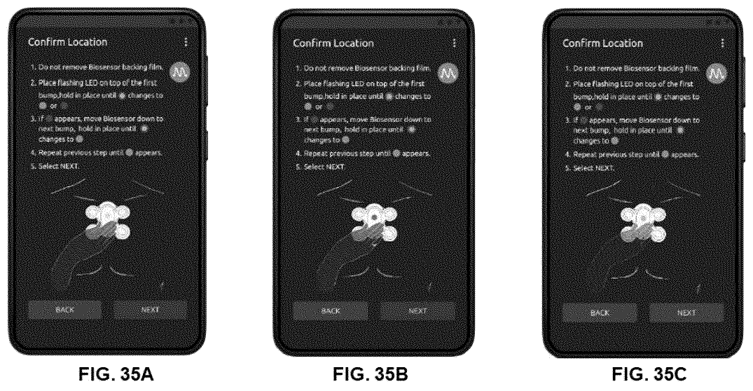

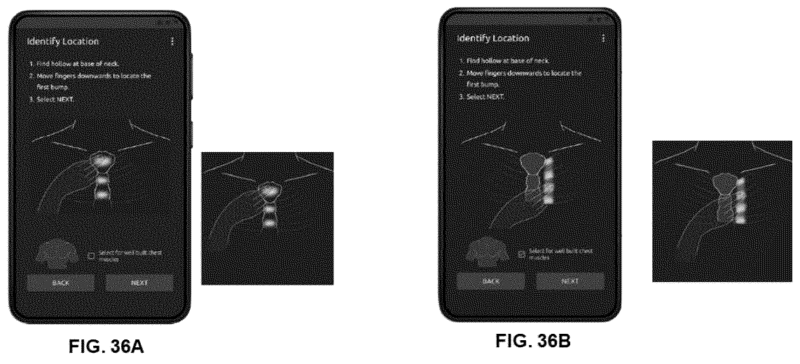

The wireless patient monitoring platform also allows patient monitoring in non-clinical settings (see 430). When patients are at home or other non-clinical environment, they may wear the patch following the guidance from respective personal applications. The guidance, as will be described in more details in accordance with FIGS. 35A, 35B, 35C, 36A, and 36B, may illustrate the correct location the patient should place the patch. The wearable patch may collect physiological signals from the patient and transmit them to the server 428 via single patient relay 426 (e.g., smart phone). The generated alerts, early warning signals/scores (EWS), and other data that is valuable to patient management may be displayed and/or further processed in end applications. For example, when patients are in a non-clinical environment, a remote patient monitoring center may be allowed to monitor the health conditions of the patients (e.g., patient list), the facilities that the patients use (e.g., facilities management), and the service providers that the patients use (e.g., service provider management) via one or more administration applications.

The monitoring applications used by health care providers in the clinical settings and administration applications used by admin staffs in the remote patient monitoring center are end applications that provide GUIs for viewers to watch the health conditions of multiple patients, and manage facilities and service providers associated with the patients. In addition, the end applications may comprise or be associated with one or more software that process physiological parameters of the patients received from the server 428, access electronic health record (EHR) of the patients, generate commercial logic, reimbursement codes and billings for administration tasks associated with patient care.

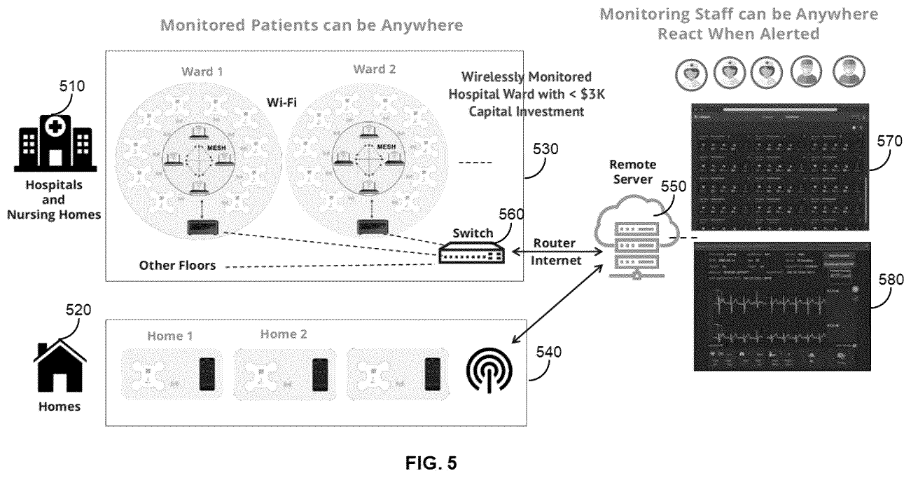

FIG. 5 illustrates an architecture of an example of patient monitoring platform. The platform as described herein allows continuous patient monitoring regardless of the physical locations of patients (see 530 and 540). As described elsewhere herein, the patient may be at a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa. A patient may be at a patient's home or outside a health care facility. A patient may be at a location that may be remote to any health care provider.

As illustrated, when admitted to any health care facility, such as hospitals and nursing homes 510, each patient may wear a patch for physiological parameter monitoring. As the hospitals and nursing homes 510 may have multiple patient wards installed with Wi-Fi access points, each patch may be wirelessly connected to a remote server 550. For example, Ward 1 and Ward 2 may be located on the same floor of a hospital, and each ward may have multiple patients wearing patches. More wards may be located on the other floors. Each patch that the patients wear may be wirelessly connected to a switch 560 and the remote server 550. The physiological parameters collected by the wearable patches may be transmitted to the remote server 550 for processing and when showing deterioration in the patient's health condition, generating alerts for clinical intervention. The processed parameters may be transmitted to one or more end applications, for example, monitoring applications 570 and 580 for health care providers in the hospitals and nursing homes 510. Hence, health care providers may watch the health conditions of multiple patients in real time and take actions when needed.

When patients are in non-clinical settings (e.g., at homes 520), the wireless platform also provides continuous patient monitoring. Patients may be mobile among home, office, and other locations. The wearable patches may be connected to the remote server 550 via wireless connections in each location. The processed parameters may be transmitted to one or more end applications, for example, administration applications for medical staff in remote monitoring centers to watch the health conditions of patients and to manage facilities as well as service providers associated with the patients.

In some instances, a patch may have an identity or identifier that may be unique to the patch. In some instances, a patch may be associated with a patient that has a unique identity or identifier. Optionally, a patch may be associated with a bodily location on a patient (e.g., central chest of patient A). In some instances, a patient may utilize a single patch. Alternatively patients may be using multiple patches simultaneously, which may or may not be differentiated from one another by identity. The data collected from a patch may be associated with the corresponding patient.

### Architecture of Wireless Patient Monitoring Platform

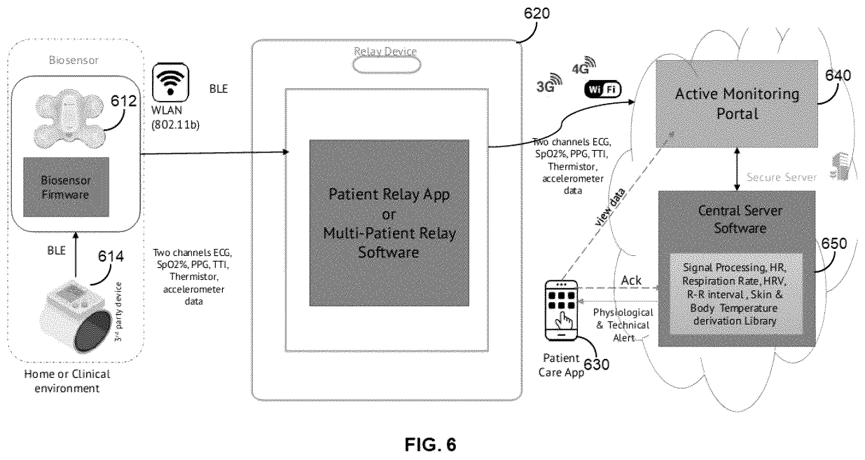

FIG. 6 illustrates an example of patient monitoring platform comprising a wearable patch with multiple radios or communication channels for wireless communication. For instance, the wearable patch may comprise a one radio that supports wireless communication between the patch with a computing device and another radio that supports wireless communication with another patch or another medical device. As illustrated in FIG. 6, for example, the wearable patch 612 comprises firmware (e.g., dual radio) that supports communication with a third party medical device 614 via Bluetooth Low Energy (BLE) and consolidates data from the patch 612 and the third party device 614. The consolidated data may be transmitted to a receiver device through another radio (e.g., Wi-Fi, MBAND, ultra-wideband (UWB)). The dual radio may also communicate with another medical device via a wireless network. Examples of the wireless network include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a direct connection between two computing devices, a peer-to-peer network, and any combinations thereof. As previously described, various communication channels may be selected based on conditions, or portion of platform with which the patch is communicating.

As illustrated in FIG. 6, data from the patch 612 and the third party device 614 may be wirelessly transmitted to a relay device 620 that comprises a patient relay application and/or multi-patient relay application. The data may be collected by a plurality of biosensors placed in the patch that measure multi-channel ECG, SpO2, pulse plethysmograph (PPG), and other physiological parameters, as well as by other sensors including thermistor and accelerometer. The relay device 620 may be positioned in clinical settings and non-clinical settings, thereby realizing the continuous patient monitoring. Compared to spot check on patients, continuous patient monitoring may comprise monitoring patients with a higher frequency and/or longer period of data collection. For example, the biosensors in the wearable patch may collect physiological parameters by second, by minute, or by hour. When the patient wears the patch, the biosensors may collect the physiological parameters of the patient throughout the use life of the patch. The patient relay application and/or multi-patient relay application may transmit the data to an active monitoring portal 640 and a central server 650 for data processing and alert generation. The active monitoring portal 640 may be located in clinical settings such as hospitals, clinics, and nursing homes, where health care providers may watch physiological parameters of the patients in real time. End applications (e.g., patient care application 630) may also receive patient data from the active monitoring portal 640. Hence, health care providers and patients may be allowed to review patient data on their portable devices. The portable device may be any device that a health care provider or patient uses, for example, phone, tablet, and laptop. The central server 650 may process patient data received from the relay device 620 and when the physiological parameters indicate the health conditions of patients are deteriorating, generate alerts to the end applications (e.g., patient care application 630) for clinical intervention. For example, the central server 650 may determine from ECG waveforms one or more of heart rate (HR), respiratory rate (RR), heart rate variability (HRV), R-R interval. The central server 650 may also generate technical alerts that indicate malfunction of the patch and transmit the alerts to the end applications (e.g., patient care application 630).

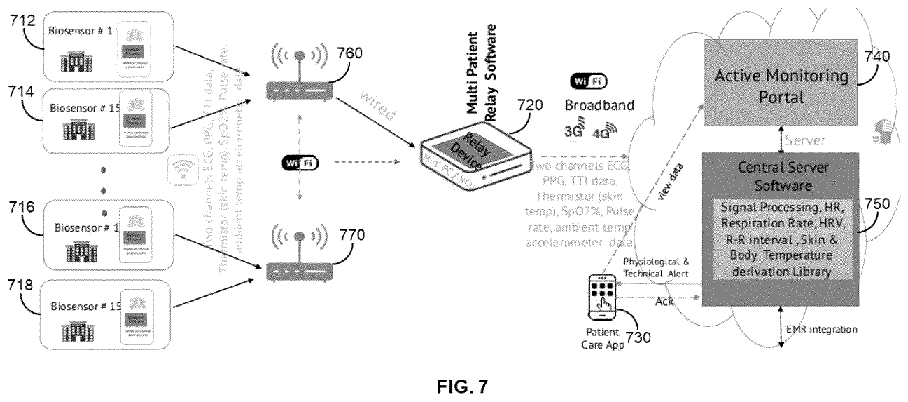

FIG. 7 illustrates an example of multi-patient monitoring platform for wireless communication. In clinical settings, hospitals may have multiple patient wards with hundreds of patients admitted. Each patient may wear a patch that comprises one or more biosensors. As illustrated, one or more patches 712, 714, 716, and 718 may be wirelessly connected to the switches 760 and 770, which in turn transmit data collected from the patches to the relay device 720. In some embodiments, the relay device may be a personal computer (PC), a network control unit (NCU), or any device that may be connected to the switches 760 and 770 in wired or wireless manner as well as to the cloud. The relay device 720 may comprise a multi-patient relay application that transmits the data to an active monitoring portal 740 and a central server 750 for data processing and alert generation. In some embodiments, the relay device may realize a plurality of communication channels that are devoted to multiple patches, such that each patch may transmit data (e.g., physiological parameters of patient). The active monitoring portal 740 may be located in clinical settings such as hospitals, clinics, and nursing homes, where health care providers may watch physiological parameters of the patients in real time. End applications (e.g., patient care application 730) may also receive patient data from the active monitoring portal 740. Hence, health care providers and patients may be allowed to review patient data on their portable devices. The central server 750 may process patient data received from the relay device 720 and when the physiological parameters indicate the health conditions of patients are deteriorating, generate alerts to the end applications (e.g., patient care application 730) for clinical intervention. The central server 750 may also generate technical alerts that indicate malfunction of the patch and transmit the alerts to the end applications (e.g., patient care application 730).

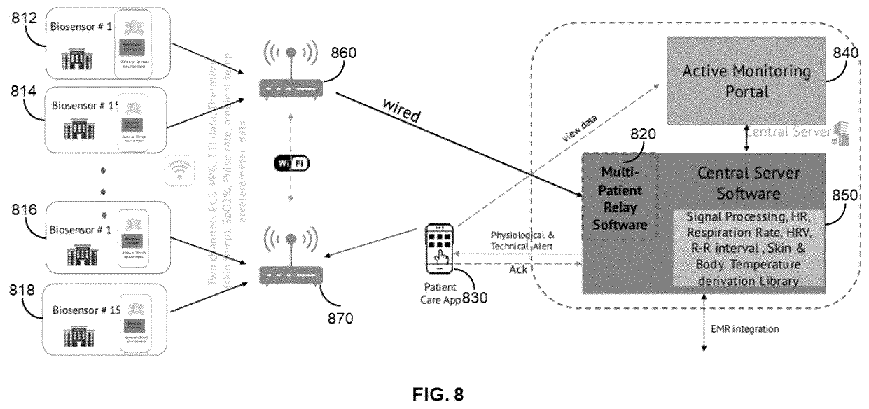

FIG. 8 illustrates another example of multi-patient monitoring platform for wireless communication. Similar to FIG. 7, one or more wearable patches 812, 814, 816, and 818 may be wirelessly connected to the switches 860 and 870. Nevertheless, no relay device is included in this example, which is different from the illustration in FIG. 7. Instead, the switch 860 may be connected to a central server 850 that comprises a multi-patient relay software 820. The switch 870 may be connected to a portable device 830 comprising a patient care application. The active monitoring portal 840 may be located in clinical settings where health care providers may watch physiological parameters of the patients in real time. End applications (e.g., patient care application) may also receive patient data from the active monitoring portal 840. Hence, health care providers and patients may be allowed to review patient data on their portable devices. The central server 850 may process patient data received from the switch 860 and when the physiological parameters indicate the health conditions of patients are deteriorating, generate alerts to the end applications (e.g., patient care application) for clinical intervention. As the degree of deviation of parameters away from clinically normal ranges indicate the severity of the health condition, the central server 850 may generate multiple levels of clinical alerts (e.g., high, medium, and low). The central server 850 may also generate technical alerts that indicate malfunction of the patch and transmit the alerts to the end applications (e.g., patient care application).

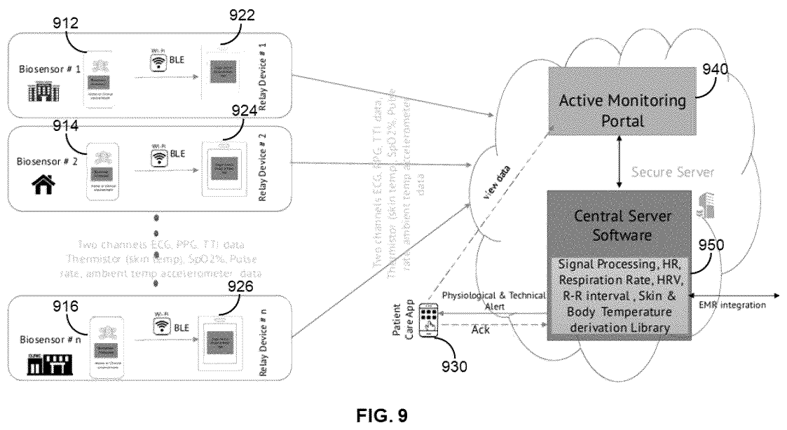

FIG. 9 illustrates another example of multi-patient monitoring platform for wireless communication. As illustrated, each of the wearable patches 912, 914, and 916may be wirelessly connected to a respective relay device 922, 924, and 926. The wireless connection may be realized using Bluetooth Low Energy (BLE). The relay devices 922, 924, and 926 may be connected to the active monitoring portal 940 and central server 950. The active monitoring portal 940 may be located in clinical settings where health care providers may watch physiological parameters of the patients in real time. End applications (e.g., patient care application) may also receive patient data from the active monitoring portal 940. Hence, health care providers and patients may be allowed to review patient data on their portable devices 930. The central server 950 may process patient data received from the relay devices 922, 924, and 926, and when the physiological parameters indicate the health conditions of patients are deteriorating, generate alerts to the end applications (e.g., patient care application) for clinical intervention. The central server 950 may also generate technical alerts that indicate malfunction of the patch and transmit the alerts to the end applications (e.g., patient care application).

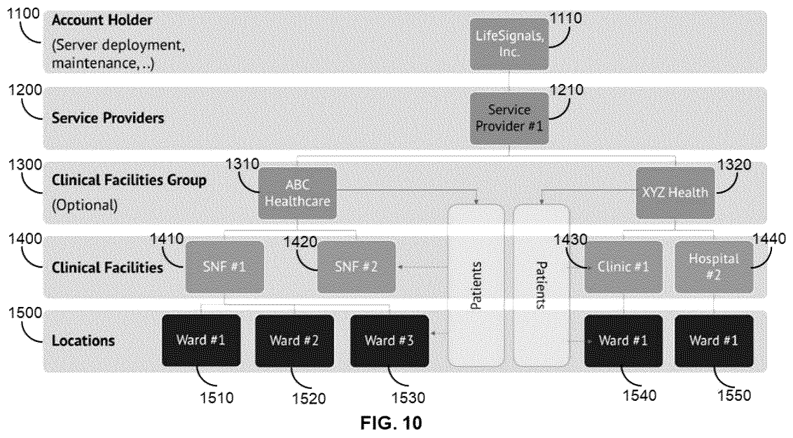

FIG. 10 illustrates user hierarchy of an example of patient monitoring platform. The user hierarchy may comprise a plurality of user levels, each user level designated with a different authority. As illustrated, account holder 1100 may comprise an entity (e.g., LifeSignals, Inc. 1110) that designs and develops the patient monitoring platform. The account holder 1100 may handle a plurality of tasks including server deployment and system maintenance. Service providers 1200 may be positioned at a lower level than the account holder 1100 within the user hierarchy. The service providers 1200 may refer to an entity that provides services in a custodial or residential setting where health, nutritional, or personal care is provided for persons receiving care. Some non-limiting examples of service providers 1200 may comprise hospitals, clinics, home health care agencies, and adult care facilities (e.g., nursing homes). In this example, service provider #1 (see 1210) is listed at this user level. Clinical facilities group 1300 may be positioned at a lower level than the service provides 1200. The clinical facilities group 1300 may be optional and integrated with the service provider level. Here, the clinical facilities group 1300 comprises two clinical providers, i.e., ABC Healthcare 1310 and XYZ Health 1320, both of which are part of the service provider #1 (see 1210). As illustrated, the clinical facilities 1400 may be positioned under the clinical facilities group 1300. The skilled nursing facility (SNF) #1 and SNF #2 (see 1410 and 1420) may be part of the ABC Healthcare 1310. The clinic #1 and hospital #2 (see 1430 and 1440) may be part of the XYZ Health 1320. The locations 1500 may be positioned further under the clinical facilities 1400, comprising a plurality of wards 1510, 1520, 1530, 1540, and 1550.

The user hierarchy as illustrated in FIG. 10 also provides a relationship between different levels. For example, the account holder LifeSignals, Inc. 1110 at the account holder level 1100 may possess or be associated with service provider #1 (see 1210) at the service providers level 1200. Service provider #1 (see 1210) may in turn possess or be associated with ABC Healthcare 1310 and XYZ Health 1320 at the clinical facilities group level 1300. As both ABC Healthcare 1310 and XYZ Health 1320 have multiple patients, the patients may be distributed to respective clinical facilities and locations. For example, patients in the ABS Healthcare 1310 may be distributed to SNF #2 and Ward #3 (see 1420 and 1530). Patient in the XYZ Health 1320 may be distributed to Clinic #1 and ward #1 (see 1430 and 1540).

As shown in FIG. 10, there are a plurality of users that may be involved in healthcare of multiple in-patients and out-patients. The patient monitoring platform provides information access to users at different level of the hierarchy, depending on the roles of the users. For example, physicians and nurses may be provided with configurable user interface to review patient data, whereas administrative staff may be provided with user interfaces to manage employees and clinical facilities. More examples of user interface will be described below in accordance with FIGS. 11-33.

### Wearable Patch

The wearable patch may be designed for monitoring physiological parameters. For example, physiological parameters can include data related to cardiac functionality, respiratory functionality, pulmonary comorbidities, and positional data. Examples of physiological data include, but not limited to, heart rate, cardiac rhythms, respiration, blood oxygenation, body temperature, conductivity, impedance, resistance, motion, orientation, position, synaptic signals, neural signals, voice signals, vision or optical signals, electrocardiography, electroatriography, electroventriculography, intracardiac electrogram, electroencephalography, electrocorticography, electromyography, electrooculography, electroretinography, electronystagmography, electroolfactography, electroantennography, electrocochleography, electrogastrography, electrogastroenterography, electroglottography, electropalatography, electroarteriography, electroblepharography, electrodermography, electrohysterography, electroneuronography, electropneumography, electrospinography, and electrovomerography.

The wearable patch may be in a small size. For example, the patch may comprise a maximum dimension equal to or smaller than about 5 centimeters (cm), 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. Alternatively, the patch may be greater than about 20 cm. The thickness of a patch may be equal or less than about 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, 4 cm, 5 cm, 6 cm, 7 cm, or 8 cm. Alternatively, the patch may have a thickness greater than about 8 cm. A patch may have a volume equal to or smaller than about 5 cm³, 10 cm³, 15 cm³, 20 cm³, 25 cm³, 30 cm³, 35 cm³, 40 cm³, 45 cm³, 50 cm³, 60 cm³, 70 cm³, 80 cm³, 90 cm³, or 100 cm³. Alternatively, the patch may have a volume greater than about 100 cm³.

The mass of the patch may be equal or less than about 1 gram, 2 grams, 3 grams, 4 grams, 5 grams, 6 grams, 7 grams, 8 grams, 9 grams, 10 grams, 11 grams, 12 grams, 13 gram, 14 grams, 15 grams, 16 grams, 17 grams, 18 grams, 19 grams, 20 grams, 21 gram, 22 grams, 23 grams, 24 grams, 25 grams, 26 grams, 27 grams, 28 grams, 29 grams, 30 grams, 31 gram, 32 grams, 33 grams, 34 grams, 35 grams, 36 grams, 37 grams, 38 grams, 39 grams, 40 grams, 41 gram, 42 grams, 43 grams, 44 grams, 45 grams, 46 grams, 47 grams, 48 grams, 49 grams, 50 grams, 60 grams, 70 grams, 80 grams, 90 grams, 100 grams, 110 grams, 120 grams, 130 grams, 140 grams, 150 grams, 160 grams, 170 grams, 180 grams, 190 grams, or 200 grams. Alternatively, the patch may have a mass greater than about 200 grams.

The patch is generally wearable. In some cases they are held onto the skin with an adhesive. In some embodiments, where an electrical signal is being measured. e.g. for ECG or EEG, some or all of the adhesive may be electrically conducting, for example comprising silver and or silver chloride. In other cases, the adhesive may be electrically non-conductive. The patch adhesive may be either wet or dry. In some embodiments, the patch may be held in place with straps or clips or may be incorporated into a piece of wearable clothing such as a hat, gloves, socks, shirt, or pants. In some embodiments, the patch may be implanted. The patches may be placed on any suitable part of the body depending on the physiological signal and the condition to be measured. For ECG monitoring, for example, a single patch can be used, the patch may be placed on the upper-chest area. For EEG monitoring, e.g. for monitoring sleep Apnea, multiple patches may be placed on the head.

A patch may comprise one or more sensors, the one or more sensors configured to monitor data (e.g., physiological, positional, etc.) from the user. For example, the number of electrodes may be 1, 2, 3, 4, 5, 6, 7, 8, 9, 10, 11, 12, 13, 14, 15, 16, 17, 18, 19, 20 or more than 20. The one or more sensors may each be a different type of sensor. Alternatively, the one or more sensors may comprise duplicate types of sensors (e.g., two temperature sensors, etc.). Sensors of the present disclosure can include, but is not limited to, electrocardiagraphy (ECG) (e.g., multi-lead ECG), tissue impedance, heart rate, heart rate variation, respiration (e.g., respiration rate and pattern), pulse oximetry (e.g., to detect peripheral capillary oxygen saturation (SpO2)), temperature, accelerometer (e.g., 3-axis), body position and motion, gyroscope, hydration, heart sound, cameras or other optical sensors, microphones or other auditory sensors, and the like.

A patch may comprise an electronic module in communication with the one or more electrodes and one or more sensors. The electronic module may be configured to receive the monitored data from the one or more electrodes and the one or more sensors. The electronic module may be operatively coupled to a wireless transmission mechanism, such as an antenna.

In some instances, the patch and components (e.g., electrodes, sensors, etc.) therein may be configured such that an orientation and placement of patch generates meaningful or high quality data. High quality data as used herein may refer to precise, accurate, interpretable, and/or reproducible data. A single patch of the present disclosure may be sufficient to collect high quality data. A placement of the patch on the human body may be of importance for obtaining a high quality data. For example, high quality data may be obtained when the patch is placed near the upper left chest or at a center of the chest. A patch placed near a center of the chest may be centered over the sternum. A patch placed near a center of the chest may be centered over the sternum and no lower than the xiphoid process.

An orientation of the patch may be of importance for obtaining high quality data. For example, when the patch is placed near the upper left chest, electrodes arranged in a normal orientation (e.g., relative to a longitudinal axis of the user) may enable acquisition of high quality data. A normal orientation as used herein may refer to an arrangement of electrodes wherein electrodes are arranged in a substantially rectangular shape relative to a longitudinal axis of the user. Electrodes arranged in a normal orientation may have a virtual line going through a LA electrode and a RA electrode, which is substantially perpendicular to a longitudinal axis of the user. Electrodes arranged in a normal orientation may have a virtual line going through a LL electrode and a RL electrode, which is substantially perpendicular to a longitudinal axis of the user. Electrodes arranged in a normal orientation may have a virtual line going through a LA and a LL electrode, which is substantially parallel to a longitudinal axis of the user. Electrodes arranged in a normal orientation may have a virtual line going through a RA and a RL electrode, which is substantially parallel to a longitudinal axis of the user. Such configuration of electrodes enables acquisition of high quality data when the patch is placed near the upper left chest. Such configuration of electrodes may enable acquisition of high quality data when the patch is placed near a center of the chest.

Patches, and/or associated software thereof, of the present disclosure may be configured to generate, and/or be capable of generating, clinical grade data or medical grade data. For example, data generated by the patches may meet the requirements of one or more accepted clinical or medical standards, models, formats, terminologies, and/or guidelines. In some instances, the standards may include different types of standards, such as measurement standards (e.g., molecular biomarkers, patient-reported outcomes, observer-reported outcomes, clinician-reported outcomes, etc.), methods standards (e.g., disease models, in vitro models, clinical trial simulation tools, etc.), and data standards (e.g., clinical data standards for certain therapeutic areas, etc.). Such standards may be created, accredited, or supported by one or more entities or consortiums, such as the Clinical Data Interchange Standards Consortiums (CDISC), Critical Path Institute (C-Path), Coalition for Accelerating Standards and therapies (CFAST), and Food and Drug Administration (FDA).

In some instances, different requirements may be associated with different therapeutic areas (e.g., cardiovascular, asthma, diabetes, pain, etc.) to qualify as clinical grade data. The patch, and/or associated software thereof, may be configured to generate clinical grade data in one or more different therapeutic areas. In some instances, the patch, and/or associated software thereof, may satisfy requirements presented by one or more open standards (e.g., CDISC Study Data Tabulation Model (SDTM) standards, Fast Healthcare Interoperability Resources (FHIR) standards, Institute of Electrical and Electronics Engineers (IEEE) standards, etc.) for clinical grade data. Alternatively or in addition, the patch, and/or associated software thereof, may satisfy requirements presented by one or more guidelines (e.g., Continua Design Guidelines). Alternatively or in addition, the patch, and/or associated software thereof, may satisfy requirements presented by one or more regulatory entities. In some instances, the patches, and/or associated software thereof, may be configured to acquire, and/or be capable of acquiring, data using an acquisition method or procedure (e.g., duration, placement of sensors, orientation of sensors, types of sensors, etc.) that satisfies one or more accepted clinical or medical standards or guidelines.

The wearable patch may comprise an electronic module. The electronic module may comprise a multi-chip module or application-specific integrated circuit (ASIC) to integrate most of the needed functions into a single module. The ASIC can be a single chip device. In some embodiments, additional components can be added to the ASIC as needed. The electronic module may comprise, or otherwise be electrically coupled to, one or more of the following: one or more of sensor interface, processing unit, and wireless communication interface. Optionally, the electronic module may comprise one or more optional units. For example, the optional units can include, but is not limited to the following: instrumentation amplifiers for ECG and/or respiration signals, signal generation units for impedance variation measurement, right leg driver circuits, antennae, transimpedance amplifiers with LED driver circuits for SpO2, micro-processors, FLASH memory, LED indicators, voltage and temperature sensors, power management units, and defibrillation (or voltage surge) protection circuits with resistors and clamping diodes. The electronic module may comprise other electronic components, such as resistors, capacitors, transcoders, and connectors, to facilitate electrical connection. Alternatively or in addition, the aforementioned components may be located elsewhere on the patch and the electronic module may be in communication with the additional components.

The electronic module may comprise a wireless communication interface. The wireless communication interface may comprise one or more of the following: a near range communication mechanism, a short range communication mechanism, and/or a long range communication mechanism. A wireless communication unit may operate on one or more of the following protocols: a Bluetooth protocol, a Wi-Fi protocol, an ultra-wide band (UWB) protocol, and a narrowband protocol. The wireless communication interface may comprise one or more radio transmitters, receivers, and/or transceivers. The radio may enable the patch to communicate with other devices through a wireless link. The radio may be configured to wirelessly transmit and/or receive data. The radio may be configured to wirelessly transmit data to external devices and/or receive data from the external devices.

The radio may be a single-mode, dual-mode, triple-mode, or quad-mode radio. The radio may utilize any known bandwidth, e.g., narrowband, wideband, ultra-wideband, broadband, etc. The radio may communicate through Wi-Fi, Bluetooth, wireless sub, and the like. In some instances, the radio may be a triple-mode hybrid radio. The triple-mode hybrid radio may utilize Wi-Fi, Medical band, and/or ultra-wideband bandwidths. The triple-mode hybrid radio may seamlessly transition between the three modes to maintain link integrity. The triple-mode hybrid radio may select a lowest power option when more than one option is available. Alternatively, the radio may be a single-mode or dual-mode radio. For example, the single-mode radio may utilize Wi-Fi. For example, the single-mode radio may utilize Medical band bandwidths. For example, the single-mode radio may utilize ultra-wideband bandwidths. In some instances, the wireless communication interface may comprise at least one narrowband radio transmitter, receiver, and/or transceiver, and at least one ultrawideband radio transmitter, receiver, and/or transceiver. The wireless communication interface may be operably coupled to one or more antennae, such as wireless transmission mechanism (e.g., one or more antennae).

The electronic module may comprise a sensor interface. The sensor interface may be operatively coupled to the one or more electrodes and the optional one or more additional sensors. In some instances, each electrode and/or sensor may be individually addressable by the sensor interface. Alternatively or in addition, a subset of electrodes and/or sensors may be addressable collectively.

The electronic module may comprise a processing unit. In some instances, the processing unit may be communicatively coupled to the sensor interface and the wireless communication interface. The processing unit can be further communicatively coupled to other electrical components, such as the power storage medium (e.g., to receive, distribute, and/or manage power, etc.) and the data storage medium (e.g., to coordinate and store application instructions and data, to coordinate and store sensor data, to coordinate and store programmable instructions of one or more processors, etc.). The power storage medium and the data storage medium, while illustrated as external to the electronic module, may in some cases be integrated in the electronic module. The processing unit can be further communicatively coupled to all or parts of the one or more optional units, such as a power management unit.

The processing unit may comprise one or more processors and a memory operably coupled to the one or more processors to execute systems and methods of the present disclosure. The processing unit may be configured to communicate with the one or more electrodes and/or the optional one or more sensors via the sensor interface, such as to receive sensor data from and/or transmit instructions to the sensors (or sensor components). The processing unit may be configured to communicate with one or more external devices (not illustrated) via the wireless communication interface, such as to receive application instructions from and/or transmit sensor data to the one or more external devices.

The processing unit may coordinate communication between an application being executed (or implemented) on an external device and a selected combination of one or more sensors. As described elsewhere herein, different sensors in the plurality of sensors may be configured to collect different types of data from the subject. Different applications executed on the one or more external devices can be configured to activate, transfer data to, receive data from, and/or otherwise utilize data from different combinations of sensors integrated in the patch. The wireless platform, and/or a selected combination of sensors therein, can be activated by an external device via one or more different applications. Different applications may utilize the different combinations of sensors of the patch to achieve one or more application-specific objectives. Such objectives can include, for example, the monitoring, tracking, and/or detection of one or more activities.

### Graphical User Interface of Patient Monitoring Platform

As described in some of the aforementioned embodiments, the patient monitoring platform may realize the continuous and wireless monitoring of multiple patients regardless of their physical locations. The backend of the patient monitoring platform may receive monitoring data from the patches worm by the patients, process and integrate it with the electronic health record (EHR) of the patients. The patient monitoring platform also provides a plurality of configurable user interfaces for different users to manage patients, users of the platform, clinical facilities, and so on.

FIG. 11A illustrates an example of graphical user interface (GUI) of a patient monitoring platform for multi patient view. As described above in accordance with FIG. when patients are admitted in hospitals, clinics, or nursing homes, health care providers may be allowed to monitor the health conditions of multiple patients via one or more monitoring applications. As illustrated, the GUI lists real-time physiological parameters of multiple patients simultaneously. For each patient, the GUI lists one or more physiological parameters including heart rate, SpO2, blood pressure, and body temperature. Some or all of the parameters may exceed a pre-determined range, the GUI may display alerts such that health care providers can take clinical intervention as needed. For example, section 1102 lists the physiological parameters of a patient with a heart rate significantly higher than a normal range. The GUI may highlight the heart rate and add a red-colored frame to the section, representing a high-level alarm that requires immediate attention from health care providers. Section 1104 lists the parameters of another patient with a body temperature relatively higher than a normal range. The GUI may highlight the body temperature and add an orange-colored frame to the section, representing a medium-level alarm. Section 1106 lists the parameters of another patient which are in normal ranges and thus, no highlight or alarm is generated.

FIG. 11B illustrates an example of GUI of a patient monitoring platform for expanded patient view. In some embodiments, the expanded patient view may comprise a zoom/hybrid view including important physiological parameters of multiple patients and details of the parameters when a particular patient is selected. As illustrated, for example, the GUI displays section 1112 including parameters of five patients. When the health care provider selects a particular patient, substantial details of his/her parameters may be displayed in section 1114, including real-time numerical values of the parameters, waveforms of parameters over a period of time, current and historical alarms if any. Alternatively or in addition, when one or more parameters of a patient triggers an alarm, the multi-patient monitoring platform may automatically display detailed information of the patient along with the alarm, such that health care providers may notice them and take immediate actions if needed.

FIG. 11C illustrates an example of GUI of a patient monitoring platform for alerts view. The GUI may display alarm information categorized by physiological parameters. As illustrated, for example, section 1122 lists an active alarm and two recent alarms of heart rate. Section 1124 lists an active alarm and two recent alarms of SpO2. Each alarm may comprise a brief description (e.g., above 120 or below 90 for heart rate), time duration (e.g., active or historical), priority (e.g., high, medium, and low), and status (e.g., whether the alarm is acknowledged by health care providers). The alerts view allows health care providers to grasp the current and historical health conditions of each patient and determine which physiological parameter(s) may show deterioration and whether clinical intervention is needed.

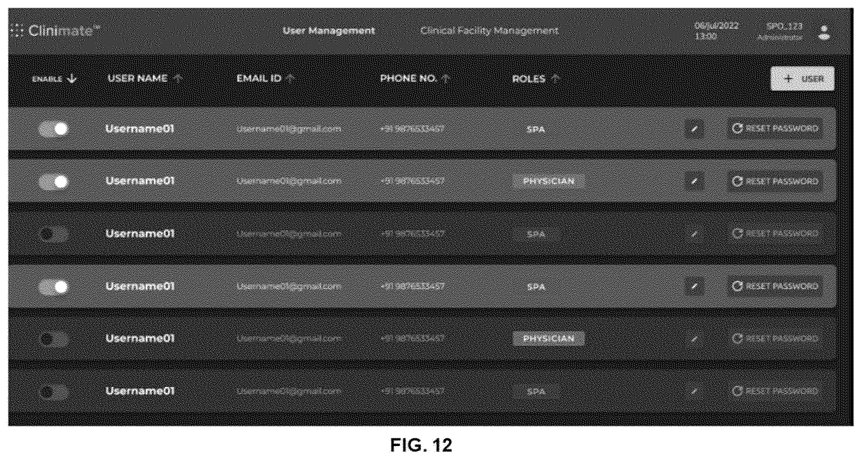

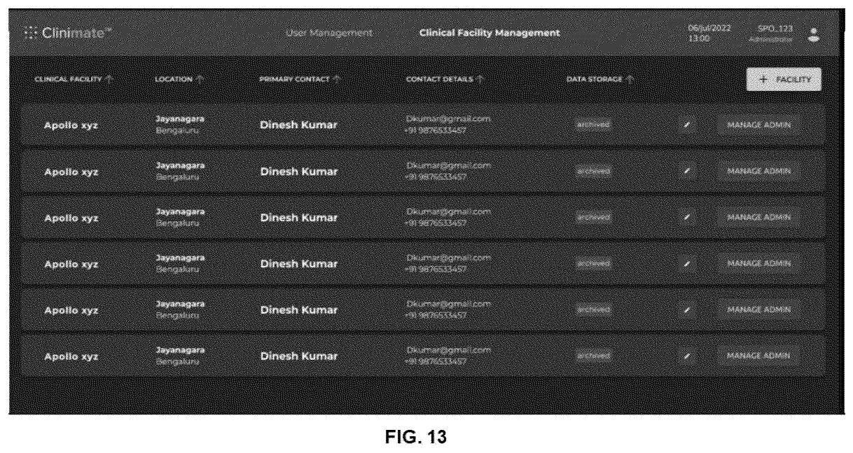

The patient monitoring platform may allow users at each hierarchical level to manage patients, facilities and other users. FIG. 12 illustrates an example of GUI of service provider admin dashboard for user management. In some embodiments, the patient monitoring platform provides the management tools to administrative staff of the service providers for user management. As illustrated, the GUI displays employees of the service providers, along with their user names, email addresses, phone numbers, roles (e.g., physician, nurses). FIG. 13 illustrates an example of GUI of service provider admin dashboard for clinical facility management. In some embodiments, the patient monitoring platform provides the management tools to administrative staff of the service providers for managing clinical facilities associated with the service provider. As illustrated, for example, the GUI lists clinical facilities with names, locations, primary contacts, and data storage.

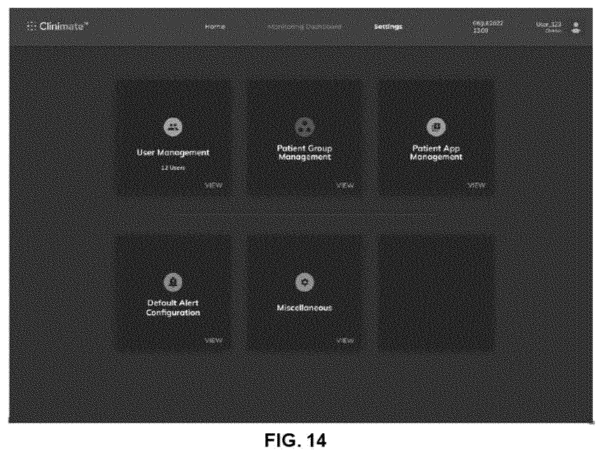

The patient monitoring platform may allow clinical facilities to manage patients, users, and facilities. Depending on the job functions of different users, the patient monitoring platform may provide customized tools. FIG. 14 illustrates an example of GUI of a settings landing page in a clinical facility admin dashboard. An administrative staff of the clinical facility may review the settings landing page and select areas of tasks he/she is assigned to, for example, user management, patient group management, patient app management, default alert configuration, and miscellaneous. Details of some of the task areas will be described as follows.

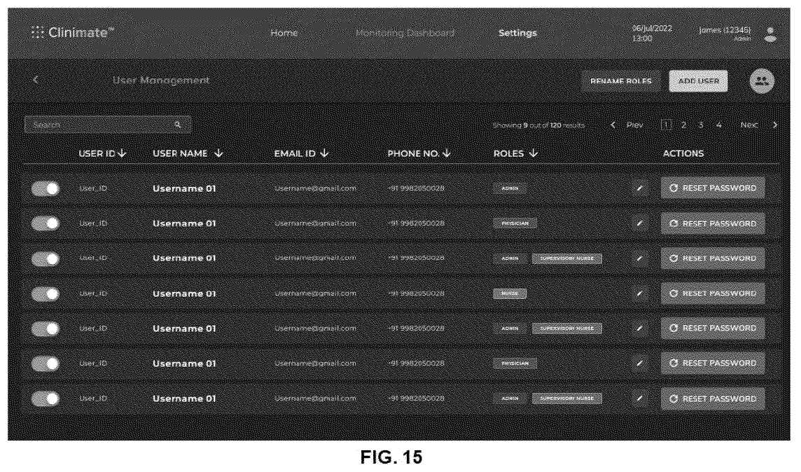

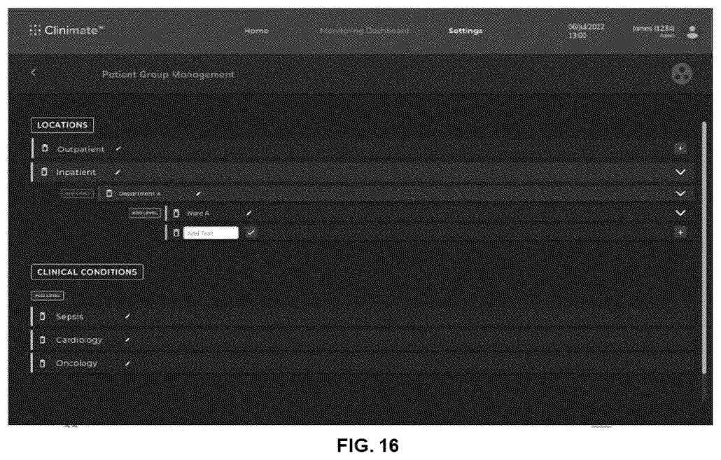

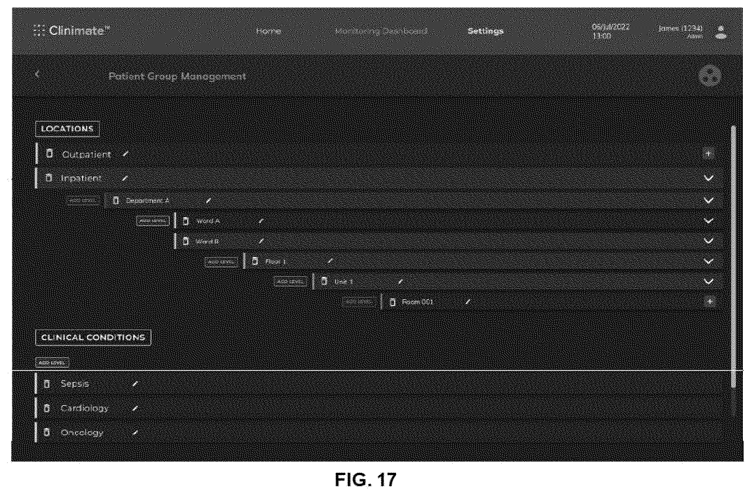

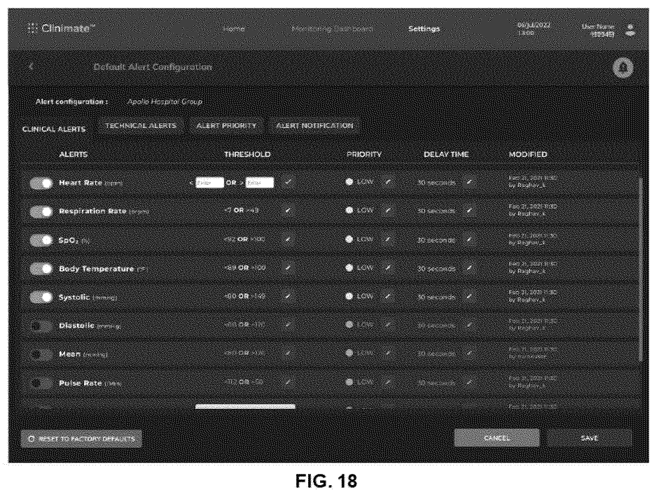

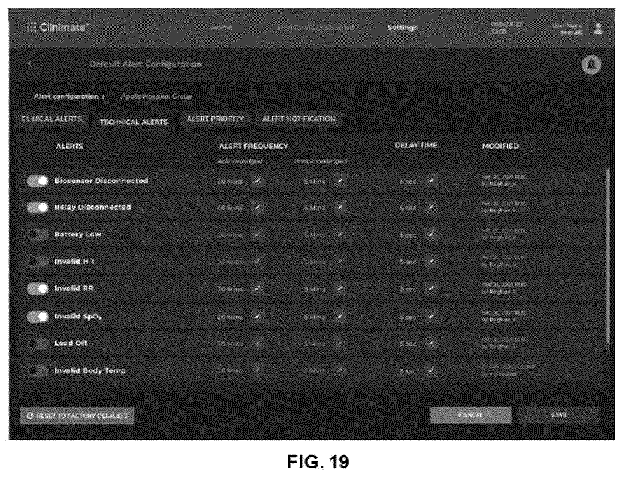

FIG. 15 illustrates an example of GUI of clinical facility admin dashboard for user management. The patient monitoring platform may allow the administrative staff to manage users of the clinical facility. As illustrated, for example, the GUI displays employees of the service providers, along with their user IDs, user names, email addresses, phone numbers, roles (e.g., physician, nurses, and admins), and actions to be taken by the staff (e.g., reset password). FIG. 16 illustrates an example of GUI of clinical facility admin dashboard for patient group customization. The patient monitoring platform may automatically group patients by locations and/or clinical conditions. Patients may be grouped as outpatient or inpatient, depending on whether he/she is admitted to this clinical facility. An inpatient may further be grouped by clinical department and/or ward. The patient monitoring platform may provide customizable configurations such that the administrative staff may group patients as needed. Alternatively or in addition, patients may be grouped by clinical conditions, for example, sepsis, conditions in cardiology and/or oncology. Automatic patient grouping with flexibility in manual tuning may facilitate patient management and improve the efficiency in patient care. FIG. 17 illustrates an example of GUI of clinical facility admin dashboard for digital twin of locations. A digital twin may refer to a virtual model that accurately reflects a physical object. Here, the patient monitoring platform may provide a digital twin of patients' location when grouping them. For example, the digital twin of locations may reflect the ward, floor, unit, and room of a particular inpatient. FIG. 18 illustrates an example of GUI of clinical facility admin dashboard for clinical alert settings. The patient monitoring platform may automatically categorize alerts by type, for example, clinical alerts and technical alerts. For example, the GUI may list a brief description of each clinical alert (e.g., heart rate, SpO2, body temperature, and systolic), one or more thresholds (e.g., higher or lower than a pre-determined value), alert priority (e.g., high, medium, and low), delay time, and history of alert modification by health care providers. FIG. 19 illustrates another example of GUI of clinical facility admin dashboard for technical alert settings. Similar to FIG. 18, the GUI may list a brief description of each technical alert (e.g., biosensor disconnected, relay disconnected, invalid respiratory rate, invalid SpO2), alert frequency, delay time, and history of alert modification.

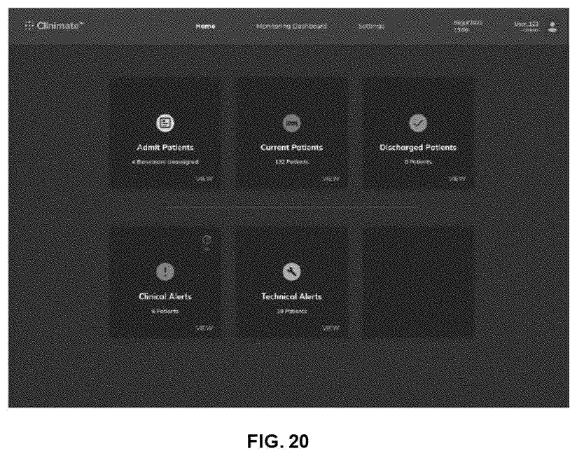

The patient monitoring platform may allow clinical facilities to manage patients. In clinical settings, each nurse and physician may often be assigned to multiple patients. The patient monitoring platform may provide a tool that allows them to manage multiple patients simultaneously. FIG. 20 illustrates an example of GUI of a landing page in a clinical facility nurse dashboard for nurses and physicians to select areas of tasks he/she is assigned to, for example, admit patients, current patients, discharge patients, clinical alerts, and technical alerts. Some of the areas of tasks will be described in detail as follows.

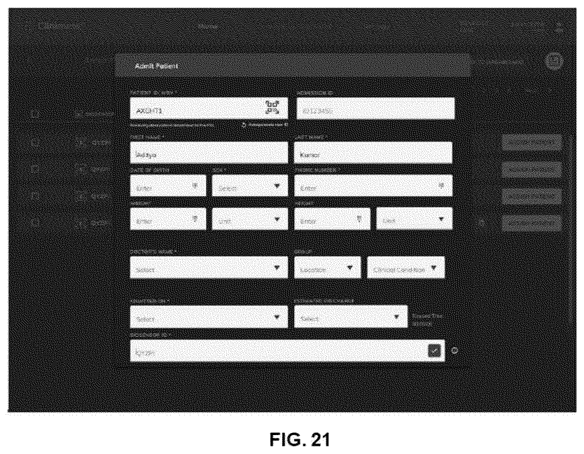

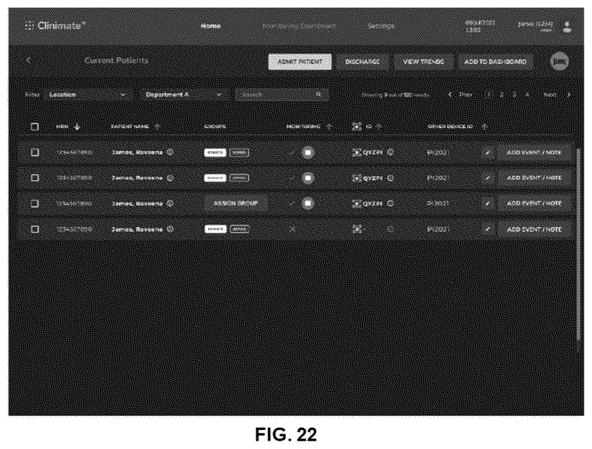

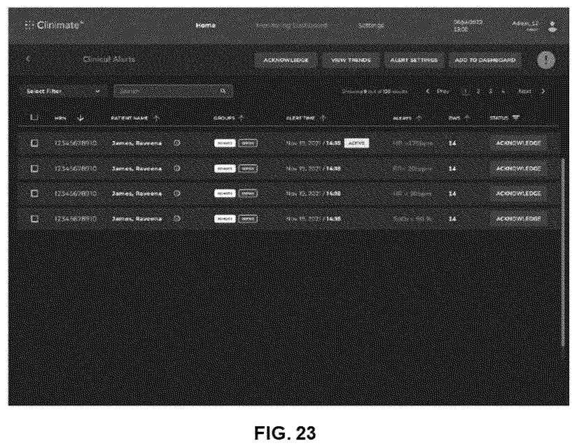

FIG. 21 illustrates an example of GUI of a clinical facility nurse dashboard for admitting patients. The GUI lists personal information of the to-be-admitted patient, including name, date of birth, phone number, medical record number (MRN), weight, and height, some or all of which may be filled in by the nurse. The GUI also lists patient ID, admission ID, date of admission, patch/biosensor ID, assigned doctor and group depending on the health condition of the patient, some or all of which may be automatically generated by the patient monitoring platform. FIG. 22 illustrates an example of GUI of a clinical facility nurse/physician dashboard for current patients view. Nurses and physicians may filter patients by groups (e.g., location, health condition). The GUI may display patient information, including medical record number (MRN), name, group (e.g., outpatient, inpatient, remote, clinical condition), whether or not under active monitoring, patch ID, and other device ID (e.g., third-party device wirelessly connected to the patch). FIG. 23 illustrates an example of GUI of a clinical facility nurse/physician dashboard for alert patients view. The GUI lists active and historical alerts of the patients, with each patient's MRN, name, group, alert time, a brief description of the alert, early warning score (EWS), and status of the alert, i.e., whether the alert is acknowledged.

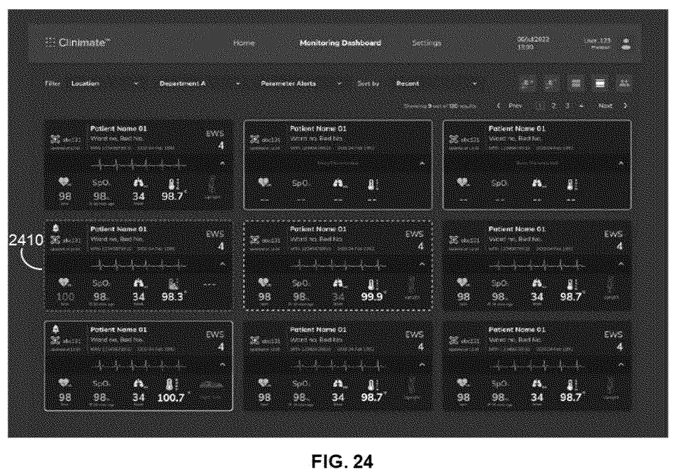

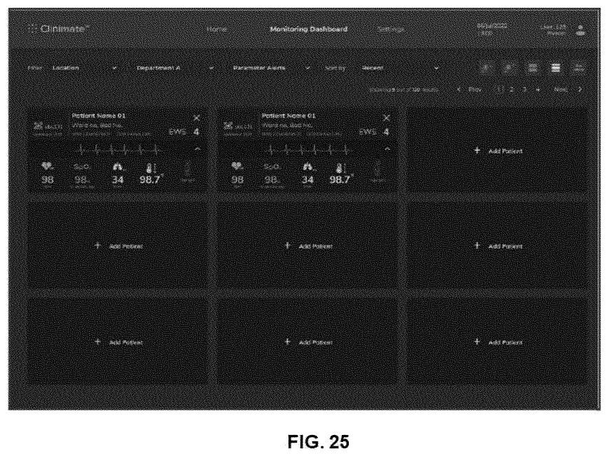

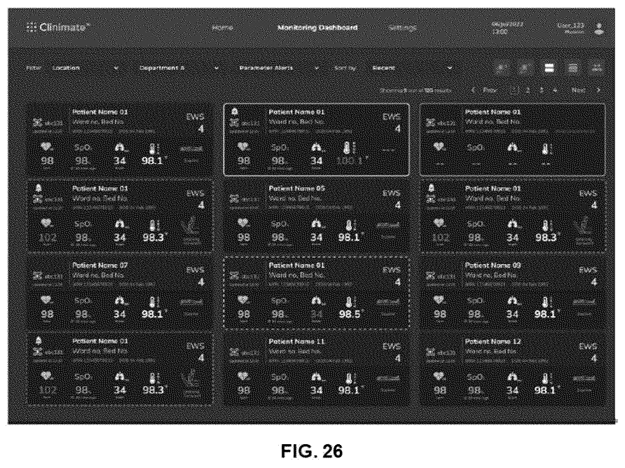

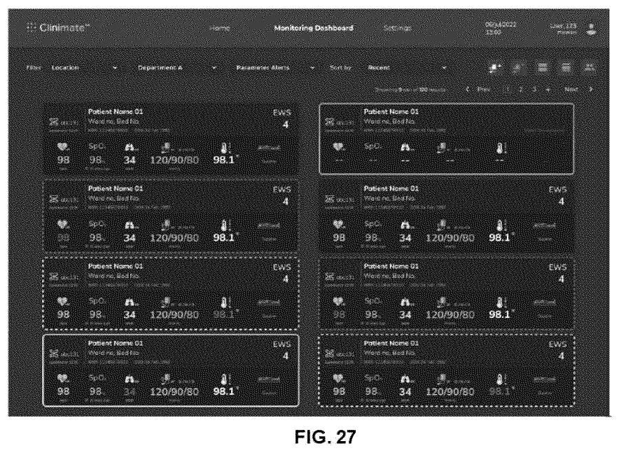

Nurses and physicians may monitor the health condition of multiple patients in real time via the patient monitoring platform. FIG. 24 illustrates an example of GUI of a nurse/physician monitoring dashboard for tile view with waveforms of physiological parameters. As illustrated, the GUI displays the parameters of multiple patients that wear patches and are under monitoring. The patients may be filtered by locations, health conditions, alert types, and so on. Each display section corresponding to an individual patient may comprise patient information, location, early warning score (EWS), and parameters that may be displayed as numerical values and/or waveforms. For example, section 2410 lists physiological parameters of a patient including heart rate, SpO2, respiratory rate, and body temperature, all of which are shown in numerical values. In addition, section 2410 includes an ECG waveform, from which nurses and physicians may determine the cardiological condition of the patient. FIG. 25 illustrates an example of GUI of a nurse/physician monitoring dashboard for tile view of custom patients. Here, nurses and physicians may be given a substantial flexibility of generating a customized patient group. Not limited by locations or health condition grouping, nurses and physicians may select patients by alternative or additional criteria, for example, patient race, age, and/or gender. FIG. 26 illustrates another example of GUI of a nurse/physician monitoring dashboard for tile view without waveforms of physiological parameters. The GUI displays the parameters of multiple patients that wear patches and are under monitoring. Different from FIG. 24, Each display section corresponding to an individual patient may comprise parameters displayed as numerical values. Excluding waveforms may realize space effectiveness. Hence, the GUI may display parameters from more patients. FIG. 27 illustrates an example of GUI of a nurse/physician monitoring dashboard for tile view with blood pressure. Different from FIGS. 24-26, here, each display section displays an additional parameter—blood pressure.

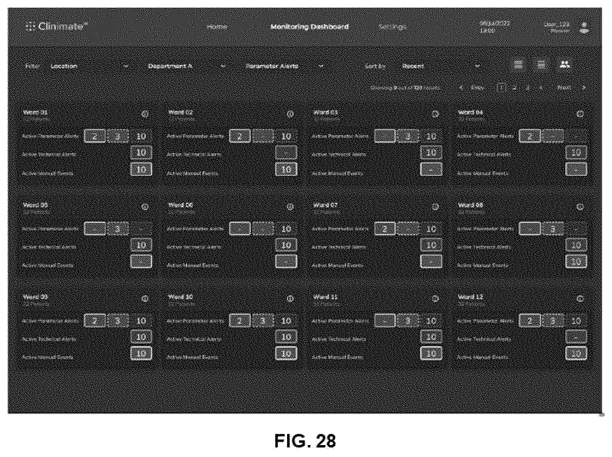

FIG. 28 illustrates an example of GUI of a nurse/physician monitoring dashboard for group view. Nurses and physicians may be allowed to monitor patients grouped by location. Instead of displaying physiological parameters, here, each display section lists alerts within a patient ward, including active clinical alerts, active technical alerts, and active manual events.

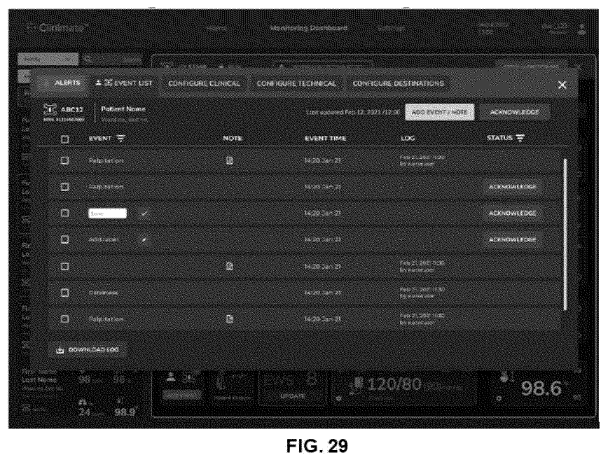

FIG. 29 illustrates an example of GUI of a nurse/physician monitoring dashboard for event listing. As illustrated, the GUI lists current and historical clinical events that a patient experienced, for example, palpitation, dizziness. Each event may comprise a brief description, event time, status, and additional notes.

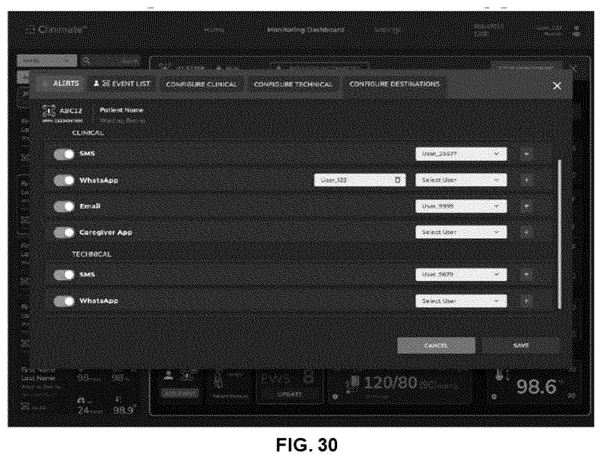

FIG. 30 illustrates an example of GUI of a nurse/physician monitoring dashboard for alert destination configuration. As described above in accordance with FIGS. 4-7, healthcare providers and patients may have portable devices with patient care applications that receive alerts. The GUI as illustrated herein provides the selection of communication channels for clinical and technical alerts, including text messages, applications, emails, and so on.

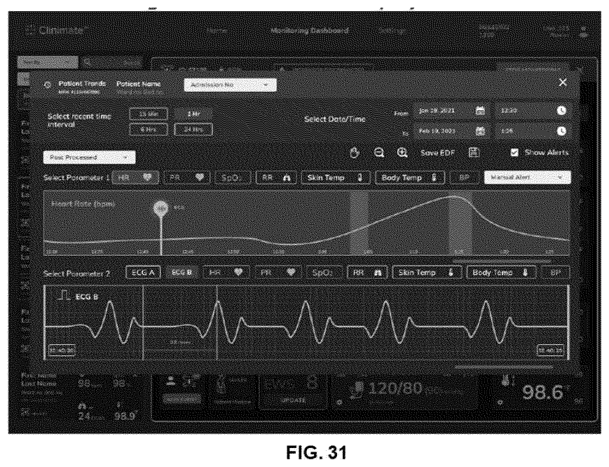

FIG. 31 illustrates an example of GUI of a nurse/physician monitoring dashboard for trend display. The GUI displays waveforms and trends of different parameters for a patient, such that physicians and nurses can review and analyze the health condition of the patient. As illustrated, the GUI displays the trend of heart rate in a duration of three hours, along with two alerts indicate the rapid increase in the heart rate.

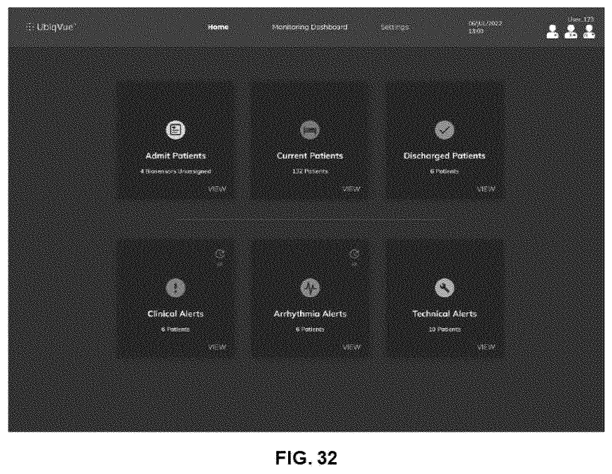

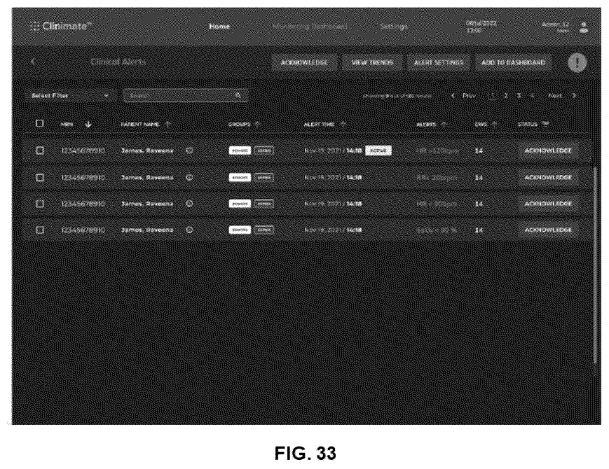

FIG. 32 illustrates an example of GUI of a settings landing page in an alert-based patient monitoring dashboard. The alert-based dashboard allows physicians and nurses to quickly navigate patient information by patient categories and alert types (e.g., clinical alerts, arrhythmia alerts, and technical alerts). FIG. 33 illustrates an example of GUI of an alert-based patient monitoring dashboard with patients with active and historical alerts.

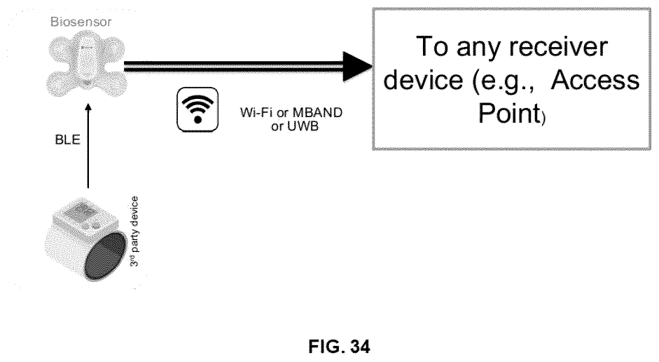

FIG. 34 illustrates an example of a wearable patch with a dual radio for wireless communication. The wearable patch may comprise a radio that supports wireless communication between the patch with a computing device and another radio that supports wireless communication with another Patch or another medical device. As illustrated in FIG. 34, for example, the wearable patch comprises a dual radio that supports communication with another medical device via Bluetooth Low Energy (BLE) and consolidates data from the patch and the medical device. The consolidated data can be transmitted to a receiver device through another radio (e.g., Wi-Fi, MBAND, ultra-wideband (UWB)). The dual radio can also communicate with another medical device via a wireless network. Examples of the wireless network include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a direct connection between two computing devices, a peer-to-peer network, and any combinations thereof.

The radio within the wearable patch may establish wireless communication with another computing device. Moreover, the radio may be used to synchronize physiological data collected by the wearable patch and another medical device. For example, a patient may wear the patch and a wireless SpO2 sensor. The wearable patch may comprise a plurality of electrocardiogram (ECG) electrodes that collect ECG signals from the patient, and the SpO2 sensor may collect pulse plethysmograph (PPG) signals. The radio in the patch may synchronize the ECG and PPG signals, which can be important for continuous non-invasive blood pressure (NIBP) monitoring using pulse arrival time (PAT) technique.

FIGS. 35A, 35B, and 35C illustrate examples of GUI that provide feedback and guide the patient to wear the patch. The GUI may be an interface of a mobile application. The wearable patch may provide visual indications for patch placement confirmation. For example, the visual indicator (e.g., LED light) may show orange-colored blinking or flashing amber (e.g., as shown in FIG. 35A), indicating the patch is assessing whether its current position on the patient body is good. When the patch is applied to a good position, the visual indicator on the patch may show a stable green color (e.g., as shown in FIG. 35C). When the position is not good, the visual indicator on the patch may show a stable red color (e.g., as shown in FIG. 35B). In addition, the GUI may display an image or animation of a body part showing a correct position of the patch, such that the patient can be guided to move the patch to the correct position. When the patient attempts to wear the patch without any assistance, he/she may find it difficult to see the visual indicator on the patch. As illustrated, the application-based GUI provides an interactive guide that enables the patient to place the wearable patch to a suitable position of the body. The application-based GUI provides a synchronized indication as the visual indicator on the patch. The patient may easily see the GUI on whether he/she places the patch to a suitable position on the body and adjust it as needed.

FIGS. 36A and 36B illustrate examples of GUI that guides the patient to wear the patch. The GUI may show graphs and/or animations that assist patients with different body shapes to identify the locations of placing the patch. As shown in FIGS. 36A and 36B, for example, the GUI shows step-by-step guidance of location identification, including “1. Find hollow at base of neck,” “2. Move fingers downwards to locate the first bump,” and “3. Select NEXT.” The GUI also illustrates the neck and chest of a human body along with a hand pointing the correct location of placing the patch. The GUI also provides flexibility to patients with different body shapes and assist them to identify the location of placing the patch. For example, patients are allowed to click “select for well-built chest muscle” as shown in FIG. 36A. The GUI provides an illustration of human body including more muscles as shown in FIG. 36B.

### Computer Systems

In some examples, the platforms, systems, media, and methods described herein may include a digital processing device, or use of the same. Any computing device described herein may be a digital processing device. In some examples, the digital processing device may include one or more hardware central processing units (CPUs) or general purpose graphics processing units (GPGPUs) that carry out the device's functions. In some examples, the digital processing device may further comprise an operating system configured to perform executable instructions. The digital processing device may be optionally connected a computer network. The digital processing device may be optionally connected to the Internet such that it accesses the World Wide Web. The digital processing device may be optionally connected to a cloud computing infrastructure. The digital processing device may be optionally connected to an intranet. The digital processing device may be optionally connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Many smartphones may be suitable for use in the system described herein. Televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some examples, the operating system may be provided by cloud computing. Suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems may include, by way of non-limiting examples, Apple TV®, Roku®, Boxee®, Google TV®, Google Chromecast®, Amazon Fire®, and Samsung® HomeSync®. Suitable video game console operating systems may include, by way of non-limiting examples, Sony® PS3®, Sony® PS4®, Microsoft® Xbox 360®, Microsoft Xbox One, Nintendo® Wii®, Nintendo® Wii U®, and Ouya®.

The device may include a storage and/or memory device. The storage and/or memory device may be one or more physical apparatuses used to store data or programs on a temporary or permanent basis. The device may be volatile memory and may require power to maintain stored information. The device may be non-volatile memory and retains stored information when the digital processing device is not powered. The non-volatile memory may comprise flash memory, dynamic random-access memory (DRAM), ferroelectric random access memory (FRAM), phase-change random access memory (PRAM).

The digital processing device may include a display to send visual information to a user. The display may be a cathode ray tube (CRT), a liquid crystal display (LCD), a thin film transistor liquid crystal display (TFT-LCD), an organic light emitting diode (OLED) display, a passive-matrix OLED (PMOLED) or active-matrix OLED (AMOLED) display, a plasma display, and/or a video projector.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. The input device may be a Kinect, Leap Motion, or the like. The input device may be a combination of devices such as those disclosed herein.

The platforms, systems, media, and methods disclosed herein may include one or more non-transitory computer readable storage media encoded with a program including instructions executable by the operating system of an optionally networked digital processing device. A computer readable storage medium may be a tangible component of a digital processing device. A computer readable storage medium is optionally removable from a digital processing device. A computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the platforms, systems, media, and methods disclosed herein may include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

A computer program may include a web application. In light of the disclosure provided herein, a web application may utilize one or more software frameworks and one or more database systems. A web application may be created upon a software framework such as Microsoft®.NET or Ruby on Rails (RoR). A web application may utilize one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL).

A computer program may include a mobile application provided to a mobile digital processing device. The mobile application may be provided to a mobile digital processing device at the time it is manufactured. The mobile application may be provided to a mobile digital processing device via the computer network described herein.

A mobile application may be created, for example, using hardware, languages, and development environments. Mobile applications may be written in various programming languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

A computer program may include a standalone application, which is a program that is run as an independent computer process, not an add-on to an existing process, e.g., not a plug-in. Standalone applications may be compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program.

The computer program may include a web browser plug-in. In computing, a plug-in may be one or more software components that add specific functionality to a larger software application. Makers of software applications support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Web browser plug-ins include, without limitation, Adobe® Flash® Player, Microsoft® Silverlight®, and Apple® QuickTime®. The toolbar may comprise one or more web browser extensions, add-ins, or add-ons. In some embodiments, the toolbar comprises one or more explorer bars, tool bands, or desk bands.

Several plug-in frameworks may be available that may enable development of plug-ins in various programming languages, including, by way of non-limiting examples, C++, Delphi, Java™, PHP, Python™, and VB .NET, or combinations thereof.

Web browsers (also called Internet browsers) are software applications, which may be configured for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror.

In some embodiments, the web browser is a mobile web browser. Mobile web browsers (also called mircrobrowsers, mini-browsers, and wireless browsers) may be configured for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

The systems, media, networks and methods described herein may include software, server, and/or database modules, or use of the same. Software modules may be created using various machines, software, and programming languages. The software modules disclosed herein are implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. The one or more software modules may comprise, by way of non-limiting examples, a web application, a mobile application, and a standalone application. In some embodiments, software modules are in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The platforms, systems, media, and methods disclosed herein may include one or more databases, or use of the same. In view of the disclosure provided herein, many databases are suitable for storage and retrieval of physiological data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. A database may be web-based. A database may be cloud computing-based. A database may be based on one or more local computer storage devices.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method for monitoring physiological data, the method comprising:
(a) contacting a surface of a subject with a patch, wherein the patch is configured to:
(1) monitor, with aid of one or more sensors operably coupled to the patch, physiological data from the subject; and
(2) receive, at an electronic module in communication with the one or more sensors, the monitored physiological data; and
(b) wirelessly transmitting the received physiological data to two or more different types of devices.

2. The method of claim 1, wherein the two or more different types of devices comprise at least two of a mobile device, a data collection device, and a patient monitor.

3. The method of claim 2, wherein the patch is configured to communicate with the data collection device, the mobile device, or the patient monitor, or any combination thereof.

4. The method of claim 3, wherein the patch is configured to communicate via Wi-Fi, MBAND, and/or UWB.

5. The method of claim 2, wherein the data collection device is further configured to communicate with one or more of: the mobile device, the patient monitor, or an external server, or any combination thereof.

6. The method of claim 5, wherein the data collection device is further configured to communicate via Wi-Fi, MBAND, and/or UWB.

7. The method of claim 3, wherein the patch is configured to communicate with the two or more devices using different communication schemes.

8. The method of claim 3, wherein the patch is configured to communicate with the two or more devices using a same communication scheme.

9. The method of claim 1, wherein the two or more different types of devices are configured to receive physiological data transmitted from a plurality of patches.

10. The method of claim 1, wherein the one or more sensors are configured to generate one or more of: electrocardiogram (ECG) data, blood saturation (SpO2) data, heart rate (HR) data, respiratory rate (RR) data, heart rate variability (HRV) data, blood pressure data, body temperature data, body posture data, body attitude data, body acceleration data, or body motion data.

11. A system for monitoring physiological data from a plurality of patients, the system comprising:
(a) a plurality of patches configured to monitor the physiological data from a plurality of patients; and
(b) a data collection device in wireless communication with one or more patches of the plurality of patches, the data collection device configured to receive the monitored physiological data from the plurality of patches, and transmit to an external server.

12. The system of claim 11, wherein the patch of the plurality of patches is configured to communicate with the data collection device via Wi-Fi.

13. The system of claim 11, wherein the plurality of patches are configured to communicate with the data collection device using a common communication scheme.

14. The system of claim 11, wherein the one or more patches of the plurality of patches is configured to measure one or more of: electrocardiogram (ECG), blood saturation (SpO2), heart rate (HR), respiratory rate (RR), heart rate variability (HRV), blood pressure, body temperature, body posture, body attitude, body acceleration, or body motion.

15. The system of claim 14, wherein the one or more patches of the plurality of patches is configured to communicate the various measured data using a common communication scheme.
